(19) United States
(12) Patent Application Publication
(10) Pub. No.: US 2022/0356517 A1
(43) Pub. Date: Nov. 10, 2022

(54) Method for Identifying Bacteria and Key Functional Genes Thereof Involved in Antimony Reduction in the Soil

(71) Applicant: Institute of Eco-environmental and Soil Sciences, Guangdong Academy of Sciences, Guangzhou (CN)

(72) Inventors: Weimin Sun, Guangzhou (CN); Baoqin Li, Guangzhou (CN); Ling Lan, Guangzhou (CN); Xiaoxu Sun, Guangzhou (CN); Miaomiao Zhang, Guangzhou (CN); Qi Wang, Guangzhou (CN); Lang Qiu, Guangzhou (CN); Xiaoyu Wang, Guangzhou (CN)

(21) Appl. No.: 17/621,430

(22) PCT Filed: Jan. 9, 2020
(86) PCT No.: PCT/CN2020/071046

§ 371 Date: Dec. 21, 2021

(30) Foreign Application Priority Data
Jun. 21, 2019 (CN) 2019 10539841.2

(51) Int. Cl. C12Q 1/6869; G16B 20/00; G16B 30/20; C12N 1/20

(52) U.S. Cl. CPC C12Q 1/6869; G16B 20/00; G16B 30/20; C12N 1/20

(57) ABSTRACT

The invention discloses a method for identifying the bacterial species and key functional genes thereof involved in antimony reduction in the soil. After consuming the original substrate by starvation culture, the sole metabolic substrate is added and the sole electron acceptor Sb(V) is provided, so that there is only one dominant electron exchange process in the system. The microorganisms metabolize and oxidize the organic substrate while coupling with the reduction of antimony, so that Sb(V) gets electrons and is reduced to Sb(III). The present invention observes the Sb(V) reduction in an anaerobic culture system of paddy soil under Sb(V) stress, and uses DNA-SIP technology to identify the phylogenic information of microorganisms that can drive the Sb(V) reduction in the culture system. The invention explores the metabolism of the antimony-reducing microorganisms and the key functional microorganisms in the paddy soil, which has great significance for understanding the antimony reduction process driven by the microorganisms, and cognizing the antimony reduction bacteria and the key functional genes.

### CROSS-REFERENCE TO RELATED APPLICATIONS

This application is the United States national phase of International Application No. PCT/CN2020/071046 filed Jan. 9, 2020, and claims priority to Chinese Patent Application No. 201910539841.2 filed Jun. 21, 2019, the disclosures of which are hereby incorporated by reference in their entirety.

### SEQUENCE LISTING

The Sequence Listing associated with this application is filed in electronic format via EFS-Web and is hereby incorporated by reference into the specification in its entirety. The name of the text file containing the Sequence Listing is 8692-2108191_ST25.txt. The size of the text file is 784 bytes, and the text file was created on Dec. 20, 2021

### TECHNICAL FIELD

The invention belongs to the field of soil microbial ecology and particularly relates to a method for identifying bacteria and key functional genes thereof involved in antimony reduction in the soil by using a stable isotope probing-metagenomics-genomics platform.

### BACKGROUND

Antimony (Sb) is a toxic metalloid belonging to group 15 of the periodic table, and distributes frequently in the lithosphere associated with As in sulfide-rich ores. As the ninth most mined metal, it's widely applied in manufacture. Recently, the harm caused by antimony has gradually attracted attention. For example, antimony has negative health effects on the human organs such as liver and kidney damage, pneumoconiosis, diarrhea, dermatitis, etc., and it may be a potential carcinogen. China is the world's largest producer of antimony, and extensive mining activities have made antimony contamination an emerging environmental concern in China.

Sb(III) and Sb(V) are the two main Sb species in the environment. Sb(III) prevails under anoxic conditions, while Sb(V) dominates in oxic environments. The mobility and toxicity of Sb strongly depend on its speciation. Sb(III) is more toxic and less soluble than Sb(V).

Microorganisms play an important role in the transformation, mobility, and bioavailability of Sb. Sb(III) can precipitate with sulfide or strongly be adsorbed by iron oxides, thereby restricting the mobility and biological toxicity of Sb(III). Therefore, microbe-mediated Sb(V) reduction combined with precipitation or adsorption might be a promising bioremediation strategy for Sb contamination. Understanding the microbe-mediated Sb transformation and the related metabolisms is of environmental importance. However, the current understanding of Sb(V)-reducing microorganisms is still very limited.

Chinese invention patent ZL 201410148322.0 discloses a method for identifying formic acid-utilizing methanogenic archaea in rice fields by DNA stable isotope probes in situ. This invention incubates the collected paddy soil samples with ¹³C-formic acid, centrifuges the total microbial DNA in the soil cultivated by ¹³C-formic acid in an ultracentrifuge and performs real-time quantitative PCR analysis and fingerprint analysis of the methanogenic archaea genes in fractions with gradient buoyancy densities to determine whether the paddy soil contains active formic acid-utilizing methanogenic archaea.

The shortcoming of the above patent is that the invention uses labeled carbon for direct identification, which is limited to target the bacteria involved in the assimilation of organic substances (such as formic acid, acetic acid, organic pollutants, etc.). However, the heavy metal(loid) compounds do not contain carbon, so it is impossible to distinguish the dissimilatory respiring bacteria involved in heavy metal/metalloid transformation.

### SUMMARY OF THE INVENTION

The molecular mechanism of Sb reduction in paddy soil is still unclear, and the microorganism responsible for Sb reduction has not yet been determined. The current understanding of Sb reduction mainly depends on limited studies of pure isolates or enriched cultures. However, the traditional isolation and cultivation may ignore some microorganisms that play an important ecological role in Sb reduction but cannot be purely cultured. To overcome this technical problem, the purpose of the present invention is to use stable isotope probing (DNA-SIP) technology to identify Sb-reducing bacteria in paddy soil.

Another purpose of the present invention is to use the stable isotope probing-metagenomics-genomics platform to reveal the key functional genes and metabolisms involved in Sb reduction, and also to provide an advanced and effective method for identifying microorganisms involved in the transformation of other heavy metal(loid)s in the future.

The purposes of the present invention are achieved through the following technical solutions:

A method uses DNA-SIP to identify the bacteria involved in Sb reduction in the soil. A microcosm is established with soil after starvation culture consuming the soil's substrate. A sole metabolic substrate (also an electron donor) is added to the microcosm, and a sole electron acceptor Sb(V) is also provided to the microcosm, so that there is only one dominant electron exchange process in the microcosm. It means that the added organic substrate is used as the electron donor, and Sb(V) is used as the electron acceptor. Microbes oxidize the organic substrates coupled with Sb reduction, resulting the generation of Sb(III) from Sb(V) by obtaining electrons; the present invention observes the reduction of Sb(V) in an anaerobic microcosm with paddy soil under Sb(V) stress and uses DNA-SIP to identify the phylogenetic information of microorganisms that can drive the Sb(V) reduction in the microcosm. The method specifically includes the following steps:

(1) sampling; adding collected soil to a mineral salt solution for anaerobic incubation until the soil background substrate is completely consumed; dividing the solution into three microcosm systems, wherein ¹³C-acetic acid and KSb(OH)₆ are added to a first microcosm system (¹³C+Sb), ¹²C-acetic acid and KSb(OH)₆ are added to a second microcosm system (¹²C+Sb), and ¹³C-acetic acid is added to a third microcosm system (¹³C) for culture;

the composition of the mineral salt solution mentioned above is: 10.55 g/L Na₂HPO₄.12H₂O, 1.5 g/L KH₂PO₄, 0.3 g/L NH₄Cl, 0.1 g/L MgCl₂, 0.00001 g/L vitamin H, 0.00002 g/L niacin, 0.0001 g/L vitamin B1, 0.00001 g/L p-aminobenzoic acid, 0.000005 g/L vitamin B5, 0.00005 g/L pyridoxamine hydrochloride, 0.00001 g/L cyanocobalamin, 10 μL/L HCl (25%, w/w), 0.0015 g/L FeCl₂.4H₂O, 0.00019 g/L CoCl₂.6H₂O, 0.0001 g/L MnCl₂.2H₂O, 0.00007 g/L ZnCl₂, 0.000024 g/L NiCl₂.6H₂O, 0.000036 g/L NaMoO₄.2H₂O, 0.000006 g/L H₃BO₃, 0.000002 g/L CuCl₂.2H₂O;

the anaerobic incubation means purging the microcosm with N₂ during the incubation;

Preferably, in each of the microcosm systems, the final concentration of ¹³C-acetic acid is 0.062 g/L, the final concentration of KSb(OH)₆ is 0.131 g/L, and the final concentration of ¹²C-acetic acid is 0.060 g/L;

(2) extracting the total DNA of soil microorganisms in the three microcosm systems where Sb reduction has been confirmed, subjecting DNA extracts to ultracentrifugation, and collecting components in different fractions respectively;

(3) determining the BD (buoyant density) value of each fraction, distinguishing heavy DNA component, medium DNA component, and light DNA component based on the BD value from high to low, purifying the components of each fraction, performing PCR amplification, selecting 1-2 components with obvious PCR amplification bands in the heavy, medium, and light centrifugal components respectively, and performing high-throughput sequencing of V4-V5 region of the 16s rRNA gene;

(4) through the high-throughput sequencing of the V4-V5 region of the 16S rRNA, comparing, analyzing and classifying the obtained sequencing data according to the existing 16S rRNA database, dividing the sequences into groups according to their similarities, wherein a group is an operational taxonomic unit (OTU) usually based on a similarity of more than 97%, and each OTU represents a different 16S rRNA sequence, that is, each OTU represents a different species of bacteria (microorganism); based on OTU analysis, analyzing the diversity of microbial communities in the samples and the abundance of different microbial species;

(5) focusing on the OTUs with high abundance in the microbial community showing in the sequencing results, and determining the ones that represent the microorganism assimilating acetic acid coupled with Sb reduction through the following steps:

Step 1: excluding the OTUs that are significantly enriched in the heavy components in the (¹³C) group. Since Sb(V) is not added to the (¹³C) group, these OTUs enriched in the heavy components are the microorganisms that assimilate ¹³C-acetic acid, but not mediate Sb reduction;

Step 2: the OTUs enriched in the medium and light components of the (¹²C+Sb) group and in the heavy component of the (¹³C+Sb) group are microorganisms assimilating acetic acid coupled with Sb reduction; the microorganisms represented by these OTUs perform acetic acid assimilation coupled with Sb reduction in the microcosm systems. Given that the acetic acid metabolized by microorganisms in the (¹³C+Sb) group is “heavier” because of ¹³C, these OTUs “moving” from the medium and light components of the (¹²C+Sb) group to the heavy component of the (¹³C+Sb) group are judged to be microorganisms assimilating acetic acid coupled with Sb reduction.

A method to identify the key functional genes of microorganisms in the soil driving the Sb reduction and metabolisms uses Sb reduction microcosm systems as enrichment culture and uses the genomic technology to evaluate the metabolism potential of Sb(V)-reducing microorganisms. Also, it focuses on the analysis of the key functional genes of microorganisms involved in the antimony reduction identified by DNA-SIP, which specifically includes the following steps:

(1) adding the soil sample as that in the above method to a mineral salt solution for anaerobic incubation until the soil background substrate is completely consumed;

(2) adding acetic acid and KSb(OH)₆ to construct a first-generation microcosm system; after all Sb(V) in the microcosm is reduced to Sb(III), diluting the first generation culture and adding acetic acid and KSb(OH)₆ to construct a second-generation culture system; similarly, after all Sb(V) in the second generation system is reduced to Sb(III), diluting the second generation culture and adding acetic acid and KSb(OH)₆ to construct a third-generation culture system; and extracting the total DNA from the soil in the second and third generation culture systems respectively;

preferably, in the culture system of each generation, the final concentration of acetic acid is 0.060 g/L, and the final concentration of KSb(OH)₆ is 0.131 g/L;

(3) performing 16S rRNA gene amplification and sequencing analysis of the total DNA of the soil in the second and third generation enrichment culture systems, comparing the results with the microbial populations identified by above mentioned DNA-SIP to confirm that the enrichment culture systems contain Sb-reducing microorganisms determined by DNA-SIP, and then proceeding to the next step of metagenomic analysis;

setting up a metagenomic library, obtaining raw sequencing Reads, performing quality control of the sequencing data, filtering low-quality data, and assembling the sequences to obtain Contigs; performing sequence comparisons, mapping the Reads of Contigs' independent data set to evaluate its abundance, performing Binning assembly on Contigs, and taking bins with integrity >90% and redundancy <10% for downstream analysis; wherein

the metagenomic library is preferably established using the Illumina Hiseq 4000 platform;

the data quality control is preferably analyzed by Trimmomatic-0.36;

the sequence assembly is preferably carried out using Megahit;

the sequence comparison is preferably carried out using Bowtie 2;

the Binning assembly is preferably carried out using the default setting of CONCOCT (version 0.4.0);

(4) analyzing the abundance of functional genes and metabolic pathways related to Sb cycling and resistance, carbon fixation, nitrogen cycling, and sulfur cycling in the metagenomic bins, and identifying the genes involved in Sb(V) reduction and the related metabolic pathways. Also, how the key elements of the biogeochemical cycle including C, N, and S influence or regulate Sb reduction are investigated. The key metabolic pathways and genes in the Sb reduction coupled with C, N, and S cyclings are studied. Accordingly, the biomolecular mechanism of Sb reduction in paddy soil is revealed.

Compared with the prior art, the present invention has the following advantages and effects:

The stable isotope probing-metagenomics-genomics platform provided by the present invention can identify the dissimilatory respiring bacteria involved in the heavy metal(loid) transformation in the soil. DNA-SIP is used to directly identify the key functional microorganisms in the community, which further serves as a guide to simplify the metagenomic structure. Then, based on the bacterial genomics, the metabolisms of the Sb-reducing microbial communities and key functional microorganisms in the paddy soil are explored. The invention is of great significance to understand the Sb reduction driven by microorganisms, Sb-reducing bacteria, and their key functional genes.

### BRIEF DESCRIPTION OF THE DRAWINGS

FIG. 1 is a schematic showing the variation of the concentrations of Sb(III) and Sb(V) in the SIP microcosm systems.

FIG. 2 is a bubble diagram of OTU relative abundance of DNA components with different representative buoyancy densities in the SIP microcosm system.

FIG. 3 is a schematic showing the relative abundance distribution of multiple bacteria with Sb reduction potential in DNA components with different buoyancy densities.

FIG. 4 is a schematic showing the phylogeny of the genome bins and read depth of microorganisms with Sb reduction potential.

FIG. 5 is a heat map related to arsenic cycling and resistance, nitrogen cycling, sulfur cycling, and carbon fixation genes in the DNA bacterial genomics of two sets of Sb reduction enrichment culture systems.

### DETAILED DESCRIPTION

Hereinafter, the present invention will be further described in detail with reference to the embodiments and the drawings, but the embodiments of the present invention are not limited thereto.

### Example 1

DNA-SIP identification of antimony reducing bacteria in paddy soil includes the following steps:

(1) Sample Collection and Processing

The soil samples were collected near an antimony mine in Hechi, Guangxi. The long-term antimony-contaminated paddy soil has become selective for microbial communities and may be enriched with antimony metabolizing microorganisms. Samples were collected in the paddy soil with a depth of 5-10 cm and transported to the laboratory at low temperatures.

(2) Establishment of DNA-SIP Microcosm Culture Systems

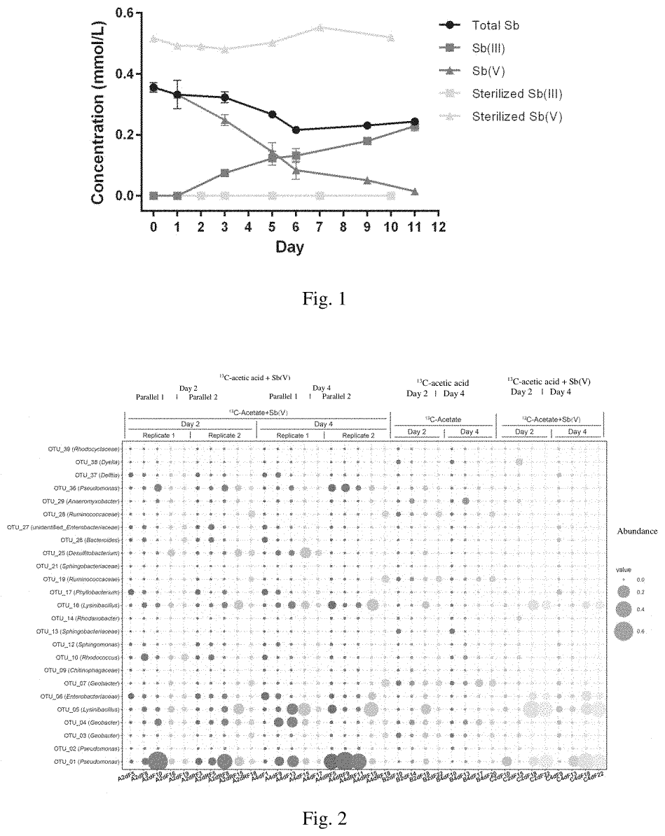

Three sets of microcosm systems were established with the collected paddy soil samples. The microcosm systems were established with 160 mL sterilized serum bottle. About 1 g of soil samples and 100 mL of mineral salt solution (Mineral Salts Medium, MSM) were added to the bottle. N₂ was purged into the system to keep the microcosm systems in an anaerobic state. The soil background substrate was completely consumed after a month of starvation culture. 0.062 g/L (final concentration) of ¹³C-acetic acid and 0.131 g/L (final concentration) of KSb(OH)₆ were added to the first (¹³C+Sb) microcosm system. 0.060 g/L (final concentration) of ¹²C-acetic acid and 0.131 g/L (final concentration) of KSb(OH)₆ were then added to the second (¹²C+Sb) microcosm system. 0.062 g/L (final concentration) of ¹³C-acetic acid was added to the third (¹³C) microcosm system. The microcosm systems were sampled on the second and fourth days of culture, and a soil DNA extraction kit was used to extract the total DNA of soil microorganisms. In addition, the concentrations of Sb(III) and Sb(V) in the solution during the entire culture process of the system were measured by a high-performance liquid chromatography-hydride generation-atomic fluorescence analyzer (HPLC-HG-AFS) (FIG. 1). It was observed that Sb(V) was reduced to Sb(III) in the microcosm system.

MSM solution composition: 10.55 g/L Na₂HPO₄.12H₂O, 1.5 g/L KH₂PO₄, 0.3 g/L NH₄Cl, 0.1 g/L MgCl₂, 0.00001 g/L vitamin H, 0.00002 g/L niacin, 0.0001 g/L vitamin B1, 0.00001 g/L p-aminobenzoic acid, 0.000005 g/L vitamin B5, 0.00005 g/L pyridoxamine hydrochloride, 0.00001 g/L cyanocobalamin, 10 μL/L HCl (25%, w/w), 0.0015 g/L FeCl₂.4H₂O, 0.00019 g/L CoCl₂.6H₂O, 0.0001 g/L MnCl₂.2H₂O, 0.00007 g/L ZnCl₂, 0.000024 g/L NiCl₂.6H₂O, 0.000036 g/L NaMoO₄.2H₂O, 0.000006 g/L H₃BO₃, 0.000002 g/L CuCl₂.2H₂O.

(3) Centrifugation of the Total DNA of Soil Microorganisms in the DNA-SIP Culture System Using an Ultra-High-Speed Centrifuge

10 μg DNA extract was placed into a 5.1 mL ultracentrifugation special quick-sealing tube. CsCl solution was added to the nearly full of the centrifuge tube. Tris-EDTA (pH 8.0) and CsCl solution were used to adjust the BD value in the centrifuge tube to 1.73 g/mL (the BD value was measured with a refractometer) followed by sealing the tube. The centrifuge tube was placed in an ultracentrifuge to centrifuge at 178,000×g at 20° C. for 48 hours. Then the centrifuge tube was taken out and placed into a fraction recovery device. A fixed flow pump was used to collect and recover different fractions of the components of the mixed solution in the centrifuge tube in the fraction recovery device, with approximately 150 μL each fraction.

(4) BD Value Determination and PCR Amplification of Each Fraction of DNA Component in Each Fraction

The BD values of the recovered components in each fraction were measured. Three groups of centrifugal components including heavy DNA centrifugal component, medium DNA centrifugal component, and light DNA centrifugal component were identified based on the BD value from high to low. Then nucleic acid precipitation aid and ethanol were used to remove CsCl by precipitation to obtain purified recovered components. Primer pairs 27F (AGAGTTTGATCMTGGCTCAG; SEQ ID NO: 1) and 1492R (GGTTACCTTGTTACGACTT; SEQ ID NO: 2) were used to perform PCR amplification on the recovered components, and representative recovered components were selected based on the BD values and PCR amplification results for the next step.

(5) Selection of DNA Components with Different Buoyancy Densities for 16S rRNA Sequencing

The method of selecting components with suitable buoyancy densities comprises: among the heavy, medium, and light components based on the BD values, 1-2 components with bright PCR amplification bands were selected, wherein 5 components (2 heavy components, 1 medium component, and 2 light components) in the first (¹³C+Sb) microcosm system were selected, 4 components (2 heavy components and 2 light components) in the second (¹²C+Sb) and third (¹³C) microcosm systems were selected respectively. High-throughput sequencing of V4-V5 regions of the 16s rRNA genes of the above components was performed.

The obtained sequencing data were compared and analyzed with the existing 16S rRNA database followed by classified into multiple groups according to their similarity. Each group conducted as an operational taxonomic unit (OTU) usually with higher than 97% of similarity. Each OTU corresponded to a different 16S rRNA sequence, that is, each OTU corresponded to a different bacterial (microbial) species. Through the OTU analysis, the diversity of the microbial community in the sample and the abundance of different microbial species were analyzed.

(6) Analysis of Antimony-Reducing Active Bacterial Communities in Paddy Soil by DNA-SIP

The sequencing results of (5) above was used for analysis. FIG. 2 shows the top 30 most abundant OTUs in the community.

In the (¹³C+Sb) microcosm system, the ¹³C-DNA enriched OTU may have the ability to assimilate acetic acid coupled with Sb(V) reduction. However, because acetic acid can also be assimilated by many bacteria that do not necessarily participate in the antimony reduction process, comparing the (¹³C+Sb) microcosm system with the (¹³C) microcosm system and comparing the (¹³C+Sb) microcosm system with the (¹²C+Sb) microcosm system is needed to eliminate interference and determine the bacteria involved in the Sb(V) reduction process.

Since there is only one dominant electron exchange process in the system involving acetic acid oxidation coupled with antimony reduction, the microorganisms participating in the reduction process of the antimony reduction system metabolized ¹³C-acetic acid, and their DNA was labeled with ¹³C. ¹³C was accumulated in the heavy component during DNA ultracentrifugation, so the phylogenetic information of OTUs representing microorganisms enriched in the heavy component of the first (¹³C+Sb) microcosm system may have the ability to assimilate acetic acid coupled with antimony reduction; in the second (¹²C+Sb) microcosm culture system, because ¹²C was added, the microbial OTUs that can assimilate acetic acid coupled with antimony reduction were enriched in the medium and light components; in the third (¹³C) microcosm culture system, the OTUs enriched in the heavy component were able to assimilate the acetic acid, but because the system did not contain antimony, these microorganisms may not be able to couple with antimony reduction.

The determination method was as follows:

Step 1: excluding the OTUs that were significantly enriched in the heavy components in the (¹³C) group. Since Sb(V) was not added to the (¹³C) group, these OTUs enriched in the heavy components were other microorganisms that assimilate ¹³C-acetic acid, but microorganisms did not involve in antimony reduction metabolism;

Step 2: after eliminating the above interference, the OTUs enriched in the medium and light components of the (¹²C+Sb) group and enriched in the heavy component of the (¹³C+Sb) group, representing microorganisms undergoing acetic acid assimilation coupled with antimony reduction were in the microcosm system. The acetic acid metabolized by microorganisms in the (¹³C+Sb) group was “heavier” because of ¹³C, then these OTUs “moving” from the medium and light components of the (¹²C+Sb) group to the heavy component of the (¹³C+Sb) group were judged to be microorganisms assimilating acetic acid coupled with antimony reduction.

After comparing the OTU results of the three groups of DNA-SIP communities in the microcosm systems, Pseudomonas, Lysinibacillus, Geobacter, and Enterobacteriaceae were found to be bacteria involved in antimony reduction metabolism.

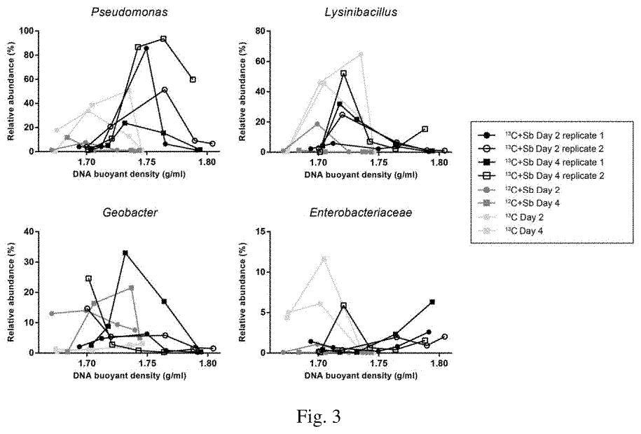

In addition, the analysis of the abundance distribution of these antimony-reducing bacteria in the DNA components with different buoyancy densities (FIG. 3) proved that all four antimony-reducing bacteria metabolize ¹³C-acetic acid so that their DNA was “heavier” and they appeared in the heavy fraction of buoyancy density. Therefore, the distribution of these antimony-reducing bacteria in the heavy fraction of buoyancy density was significantly different from those in the light fraction.

### Example 2

The metagenomic-single bacteria draft assembly revealed the functional genes related to antimony reduction includes the following steps:

(1) Establishment of Sb(V) Reduction Enrichment Culture System A first-generation microcosm system was established using the paddy soil samples collected in Example 1. The microcosm system was established in a 100 mL sterilized serum bottle. About 5 g of soil and 50 mL of MSM solution were added to the bottle, and N₂ was purged into the bottle to keep the microcosm systems in an anaerobic state. After a month of starvation culture, the soil background substrate was consumed.

MSM solution composition: 10.55 g/L Na₂HPO₄.12H₂O, 1.5 g/L KH₂PO₄, 0.3 g/L NH₄Cl, 0.1 g/L MgCl₂, 0.00001 g/L vitamin H, 0.00002 g/L niacin, 0.0001 g/L vitamin B1, 0.00001 g/L p-aminobenzoic acid, 0.000005 g/L vitamin B5, 0.00005 g/L pyridoxamine hydrochloride, 0.00001 g/L cyanocobalamin, 10 μL/L HCl (25%, w/w), 0.0015 g/L FeCl₂.4H₂O, 0.00019 g/L CoCl₂.6H₂O, 0.0001 g/L MnCl₂.2H₂O, 0.00007 g/L ZnCl₂, 0.000024 g/L NiCl₂.6H₂O, 0.000036 g/L NaMoO₄.2H₂O, 0.000006 g/L H₃BO₃, 0.000002 g/L CuCl₂.2H₂O.

(2) Dilution and Transfer of Two Generations of Sb(V) Reduction Enrichment Culture Systems, and Total DNA Extraction for Each Generation of Culture System

0.060 g/L (final concentration) of acetic acid and 0.131 g/L (final concentration) of KSb(OH)₆ were added for culturing. When all Sb(V) in the system was reduced to Sb(III), the first-generation culture system was diluted at a ratio of 1:10 and transferred to a second-generation culture system. 0.060 g/L (final concentration) of acetic acid and 0.131 g/L (final concentration) of KSb(OH)₆ were added to the second-generation microcosm system for further culturing. Similarly, when all Sb(V) in the second-generation system was reduced to Sb(III), the second-generation culture system was diluted at a ratio of 1:10 and transferred to the third-generation culture system. 0.060 g/L (final concentration) of acetic acid and 0.131 g/L (final concentration) of KSb(OH)₆ were added to the third-generation microcosm system for further culturing. The total DNA of the soil in the second and third generation culture systems was extracted respectively.

(3) Metagenomic Analysis of DNA Samples Obtained from the Enrichment Culture, and Draft Metagenome-Assembled Genomes

The total DNA of the soil in the second and third generation culture systems was analyzed by 16S rRNA gene amplification and sequencing and the results were compared with that of the aforementioned DNA-SIP microbial populations to confirm that the communities of the enrichment culture systems contained antimony-reducing microorganisms judged by DNA-SIP. The metagenomic analysis was conducted.

A metagenomic library was established on the Illumina Hiseq 4000 platform to obtain the original sequencing Reads. Trimmomatic-0.36 was used for sequencing data quality control. Low-quality data was filtered, and then Megahit was used for sequence assembly to obtain Contigs. Bowtie 2 was used for sequence comparison. The Reads of Contigs' independent data set was mapped to evaluate its abundance, followed by using the default setting of CONCOCT (version 0.4.0) for Binning assembly of Contigs. Bins with integrity >90% and redundancy <10% were collected to perform downstream analysis.

(4) Analysis Revealing Functional Genes Related to Antimony Reduction Metabolism by Metagenomic-Single Bacteria Draft Assembly

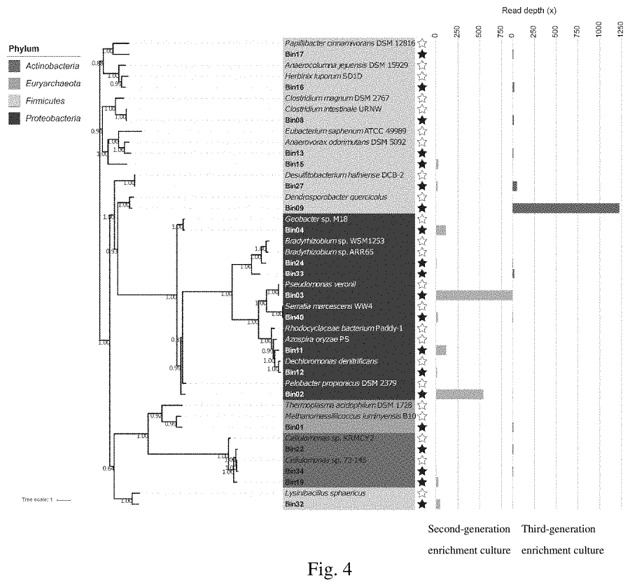

Metagenomic analysis was used to study the metabolic potential of Sb(V) reducing microbial communities, especially for those antimony reducing bacteria identified by DNA-SIP. After analysis, 20 high-quality bins were obtained from metagenomic data. These bins belonged to the four categories including Actinobacteria, Euryarchaeota, Firmicutes, and Proteobacteria (FIG. 4). In these bins, 4 kinds of antimony reducing bacteria (integrity >95%, pollution degree <5%) inferred from DNA-SIP were also detected. The abundance of functional genes and metabolic pathways related to carbon fixation, nitrogen cycle, sulfur cycle, antimony cycle, and resistance in metagenomic bins were analyzed. In addition, there was no functional gene related to the antimony cycle in the existing database, but since antimony and arsenic have similar chemical structures, studies have suggested that bacteria can use similar metabolic pathways to transform antimony and arsenic, so the gene for the arsenic cycle was used as reference. Accordingly, genes and metabolic pathways related to Sb(V) reduction were obtained, and how the key elements of biogeochemical cycles including C, N, and S influence or regulate antimony reduction was learned. Also, key genes and key metabolic pathways involving C, N, and S cycles coupled with antimony reduction were explored, thereby understanding the biomolecular mechanism of antimony reduction in paddy soil.

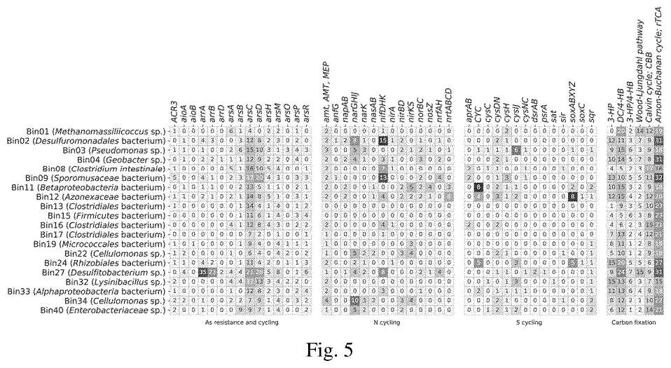

The results show (FIG. 5) that the gene for arsenic cycle arsC existed in 20 bins, and the bins belonging to Desulfitobacterium have the highest open reading frames (ORFs) in the genes arrA and arrB. Among the four deduced antimony-reducing bacteria in DNA-SIP, the bins belonging to Geobacter contained the arrABD gene, the bins belonging to Pseudomonas and Enterobacteriaceae only contained the arrA gene, and the bin belonging to Lysinibacillus contained the arsC gene. In addition, the arsenic resistant gene arsHDR existed in many bins. Except for Enterobacteriaceae, most bins contained the arsenite methyl transfer gene arsM. In the genetic analysis of metabolic pathways related to carbon, nitrogen, and sulfur, rTCA is the most abundant carbon-fixed metabolic pathway, nifDHK is the most abundant nitrogen cycle metabolic pathway, and cyclL is the most abundant sulfur cycle metabolic pathway, which proves that the metabolic pathways of C, N, and S play a dominant role in the antimony reduction system and have an important influence on the microbial conversion process of antimony.

This result revealed the potential antimony-reducing bacteria and their metabolic pathways in paddy soils and expands the current understanding of the ecological functions of antimony-reducing bacteria in paddy soils and the geochemical cycle of antimony-driven by microorganisms.

The above-mentioned embodiments are preferred embodiments of the present invention, but the embodiments of the present invention are not limited by the above-mentioned embodiments, and any other changes, modifications, substitutions, combinations, and simplifications made without departing from the spirit and principle of the present invention all should be equivalent replacement methods, and they are all included in the protection scope of the present invention.

1. A method of using stable isotope probing (DNA-SIP) to identify a bacterial species involved in antimony reduction in a soil sample, the method comprising the steps of:
(1) (i) adding to the soil sample a mineral salt solution for anaerobic culture until a background substrate of the soil sample is consumed; (ii) dividing the solution into three microcosm systems, wherein ¹³C-acetic acid and KSb(OH)₆ are added to a first (¹³C+Sb) microcosm system, ¹²C-acetic acid and KSb(OH)₆ are added to a second (¹²C+Sb) microcosm system, and ¹³C-acetic acid is added to a third (¹³C) microcosm system for culture;
(2) (i) extracting total DNA in the three microcosm systems wherein antimony reduction is confirmed, (ii) subjecting the DNA extracts to ultra-high-speed centrifuge, and (iii) collecting centrifugal components in different fractions respectively;
(3) (i) determining a buoyant density (BP) value of each fraction of the centrifugal components, (ii) distinguishing a heavy DNA centrifugal component, a medium DNA centrifugal component, and a light DNA centrifugal component based on the BD value from high to low, (iii) purifying the centrifugal components of each fraction, (iv) performing PCR amplification, (v) selecting 1-2 components with bright PCR amplification bands respectively in the heavy, medium, and light centrifugal components, (vi) and performing high-throughput sequencing of V4-V5 region of the 16s rRNA gene;
(4) (i) comparing, analysing, and classifying, through the high-throughput sequencing of the V4-V5 region of the 16S rRNA gene, the obtained sequencing data according to the existing 16S rRNA database, and (ii) dividing the sequences into a plurality of operational taxonomic units (OTUs) according to their similarities; and
(5) (i) focusing on the OTUs with high abundance in the microbial community of the sequencing results, and (ii) confirming the OTUs enriched in the medium and light components of the (¹²C+Sb) group and enriched in the heavy component of the (¹³C+Sb) group to be microorganisms assimilating acetic acid coupled with antimony reduction.

2. The method according to claim 1, wherein in each of the microcosm systems, the final concentration of ¹³C-acetic acid is 0.062 g/L, the final concentration of KSb(OH)₆ is 0.131 g/L, and the final concentration of ¹²C-acetic acid is 0.060 g/L.

3. The method according to claim 1, wherein the similarities in step (4) are higher than 97%.

4. A method for identifying functional genes of microorganisms in a soil sample responsible for antimony reduction process and metabolic pathways thereof, the method comprising the steps of:
(1) adding the soil sample to a mineral salt solution for anaerobic culture until a background substrate of the soil sample is completely consumed;
(2) (i) adding acetic acid and KSb(OH)₆ for culture to form a first-generation microcosm culture system; (ii) diluting, after all Sb(V) in the system is reduced to Sb(III), the first generation culture system and adding acetic acid and KSb(OH)₆ for further culture to form a second-generation culture system; (iii) diluting, after all Sb(V) in the second generation system is reduced to Sb(III), the second generation culture system and adding acetic acid and KSb(OH)₆ for further culture to form a third-generation culture system; and (iv) extracting total DNA from the soil sample in the second and third generation culture systems respectively;
(3) (i) performing 16S rRNA gene amplification and sequencing analysis on the total DNA of the soil sample in the second and third generation enrichment culture systems, (ii) comparing the results with DNA-SIP microbial population to confirm that the communities in the enrichment culture systems contain antimony-reducing microorganisms determined by DNA-SIP;
(iv) setting up a metagenomic library, obtaining original sequencing Reads, performing sequencing data quality control, filtering low-quality data, and assembling the sequence to obtain Contigs; performing sequence comparisons, mapping the Reads of Contigs' independent data set to evaluate its abundance, performing Binning assembly on Contigs, and taking bins with integrity >90% and redundancy <10%; for downstream analysis; and
(4) (i) analyzing the abundance of functional genes and metabolic pathways related to antimony cycle and resistance, carbon fixation, nitrogen cycle, and sulfur cycle in the metagenomic bins, and (ii) identifying the genes related to Sb(V) reduction metabolism and the metabolic pathways.

5. The method according to claim 4, wherein each of the microcosm culture systems in step (2), the final concentration of acetic acid is 0.060 g/L, and the final concentration of KSb(OH)₆ is 0.131 g/L.

6. The method according to claim 4, wherein
the metagenomic library is established using the Illumina Hiseq 4000 platform; and wherein
the data quality control is analyzed by Trimmomatic-0.36.

7. The method according to claim 4, wherein
the sequence assembly is carried out using Megahit; and
the sequence comparison is carried out using Bowtie 2.

8. The method according to claim 4, wherein
the Binning assembly is carried out using the default setting of CONCOCT.

9. The method according to claim 1, wherein the mineral salt solution comprises 10.55 g/L Na₂HPO₄.12H₂O, 1.5 g/L KH₂PO₄, 0.3 g/L NH₄Cl, 0.1 g/L MgCl₂, 0.00001 g/L vitamin H, 0.00002 g/L niacin, 0.0001 g/L vitamin B1, 0.00001 g/L p-aminobenzoic acid, 0.000005 g/L vitamin B5, 0.00005 g/L pyridoxamine hydrochloride, 0.00001 g/L cyanocobalamin, 10 μL/L HCl (25%, w/w), 0.0015 g/L FeCl₂.4H₂O, 0.00019 g/L CoCl₂.6H₂O, 0.0001 g/L MnCl₂.2H₂O, 0.00007 g/L ZnCl₂, 0.000024 g/L NiCl₂.6H₂O, 0.000036 g/L NaMoO₄.2H₂O, 0.000006 g/L H₃BO₃, and 0.000002 g/L CuCl₂.2H₂O.

10. The method according to claim 1, wherein the anaerobic culture in step (1) comprises purging the culture system with N₂ during the culture.

11. The method according to claim 4, wherein the mineral salt solution comprises 10.55 g/L Na₂HPO₄.12H₂O, 1.5 g/L KH₂PO₄, 0.3 g/L NH₄Cl, 0.1 g/L MgCl₂, 0.00001 g/L vitamin H, 0.00002 g/L niacin, 0.0001 g/L vitamin B1, 0.00001 g/L p-aminobenzoic acid, 0.000005 g/L vitamin B5, 0.00005 g/L pyridoxamine hydrochloride, 0.00001 g/L cyanocobalamin, 10 μL/L HCl (25%, w/w), 0.0015 g/L FeCl₂.4H₂O, 0.00019 g/L CoCl₂.6H₂O, 0.0001 g/L MnCl₂.2H₂O, 0.00007 g/L ZnCl₂, 0.000024 g/L NiCl₂.6H₂O, 0.000036 g/L NaMoO₄.2H₂O, 0.000006 g/L H₃BO₃, and 0.000002 g/L CuCl₂.2H₂O.

12. The method according to claim 4, wherein the anaerobic culture in step (1) comprises purging the culture system with N₂ during the culture.


SEQUENCE LISTING (2 sequences)

SEQ ID NO: 1 - DNA, 20 bp, Artificial Sequence
  Forward Primer
agagtttgatcmtggctcag

SEQ ID NO: 2 - DNA, 19 bp, Artificial Sequence
  Reverse Primer
ggttaccttgttacgactt